(19) United States
(12) Patent Application Publication
(10) Pub. No.: US 2007/0135390 A1
(43) Pub. Date: Jun. 14, 2007

(54) Phosphate derivatives

(76) Inventors: micheal simon West, belgrave south (AU); David Kannar, Belgrave south (AU)

Correspondence Address: REED SMITH LLP, P.O. BOX 488, PITTSBURGH, PA, 15230-0488, US

(21) Appl. No.: 10/551,200

(22) PCT Filed: Apr. 14, 2004
(86) PCT No.: PCT/AU04/00491

§ 371 Date: Aug. 31, 2006

(30) Foreign Application Priority Data
Apr. 15, 2003 (AU) 2003901815

(51) Int. Cl. A61K 31/66; C07F 9/02; C07J 51/00

(52) U.S. Cl. 514/114; 514/130; 558/190; 558/194; 552/506

(57) ABSTRACT

According to the invention, there is provided a phosphate derivative of a phenolic hydroxy compound comprising the reaction product of the following steps: (d) reacting the phenolic hydroxy compound with an alkyl α:ω dialdehyde or a sugar-like polyhydroxy dialdehyde to form a hemiacetal; (e) reducing the terminal aldehyde group on the product from step (a) to a hydroxyl group; and (f) phosphorylating the hydroxyl group formed in step (b) to produce a phosphate derivative of the phenolic hydroxy compound.

### FIELD OF THE INVENTION

The invention relates to a phosphate derivative of a phenolic hydroxy compound and a method for producing that derivative.

### BACKGROUND OF THE INVENTION

In this specification, where a document, act or item of knowledge is referred to or discussed, this reference or discussion is not an admission that the document, act or item of knowledge or any combination thereof was at the priority date part of common general knowledge; or known to be relevant to an attempt to solve any problem with which this specification is concerned.

Whilst the following discussion relates to the potential use of the phosphate derivative of the invention in the delivery of active compounds in anaesthetics, it will be understood that the invention may also have application to other compounds containing phenolic hydroxyl groups where improved water solubility, rapid activity or improved delivery is desired, for example, adrenaline (CAS 51-43-4 & 9945-6) and analgesics (CAS 36322-90-4).

An ideal anaesthetic drug would induce anesthesia smoothly and quickly, then permit rapid patient recovery upon cessation. The drug would also be safe to use and free of side effects, but as no single agent possesses all these attributes, combinations of drugs are often used in modern practice.

Propofol is an extremely important intravenous induction agent as it produces anesthesia at a rate similar to intravenous barbiturates but recovery is more rapid. Patients report feeling better in the immediate postoperative period and are able to ambulate sooner in comparison to other agents. Postoperative vomiting and nausea is uncommon as propofol is reported to have anti-emetic actions. For these reasons propofol is a popular drug, especially in day surgery where it is used both as an induction and maintenance anesthetic.

An important disadvantage of propofol arises from its lipid solubility, requiring the compound to be delivered in other more soluble lipidic carriers that improve dissolution such as medium chain length triglyceride (Cremophor), oil in water emulsion (Intralipid), polyoxyl 35 castor oil (hydrogenated castor oil) or other lipidic emulsion systems.

Hypersensitivity reactions have been reported with propofol. These include hypotension, flushing and bronchospasm, that are largely thought to be due to the lipid vehicle Cremaphor. A potential alternative approach is to use propofol phosphate which is a water soluble derivative of propofol. Intravenous administration of propofol phosphate would be expected to convert to the parent compound via the action of plasma and tissue phosphatases such as alkaline phosphatase. In vitro use of propofol phosphate does not however induce anesthesia and does not release the parent drug because the phosphate group is slow to hydrolyse.

Therefore, there is still a need for further derivatives of phenolic hydroxy compounds which might be used to enhance delivery of certain active compounds.

### SUMMARY OF THE INVENTION

According to a first aspect of the invention, there is provided a phosphate derivative of a phenolic hydroxy compound comprising the reaction product of the following steps:
(a) reacting the phenolic hydroxy compound with an alkyl o:o) dialdehyde or a sugar-like polyhydroxy dialdehyde to form a hemiacetal;
(b) reducing the terminal aldehyde group on the product from step (a) to a hydroxyl group; and
(c) phosphorylating the hydroxyl group formed in step (b).

The following Reaction Schemes 1 and 2 illustrate the three reaction steps according to the first aspect of the invention. In both of the schemes, R¹, R², R³, R⁴ and R⁵ may each independently be chosen from H or an alkyl group. In Reaction Scheme l, n and m are independently in the range of 0 to 8. In Reaction Scheme 2, R⁶, R⁷ and R⁸ can each independently be H or OH.

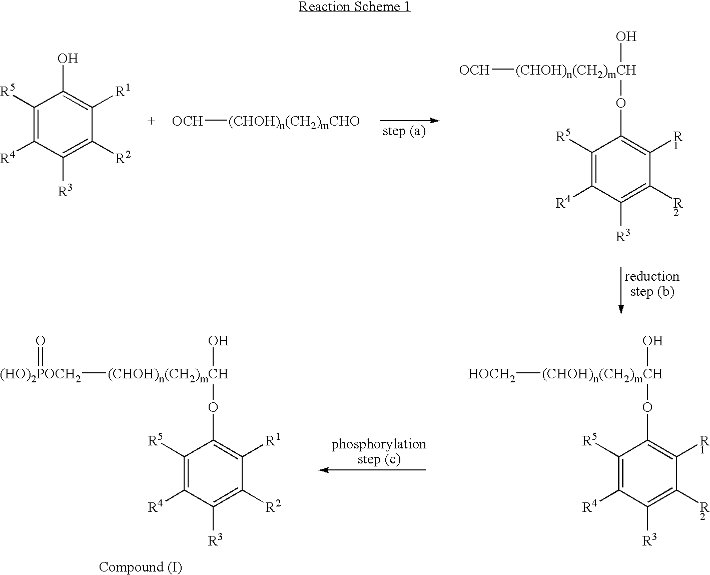

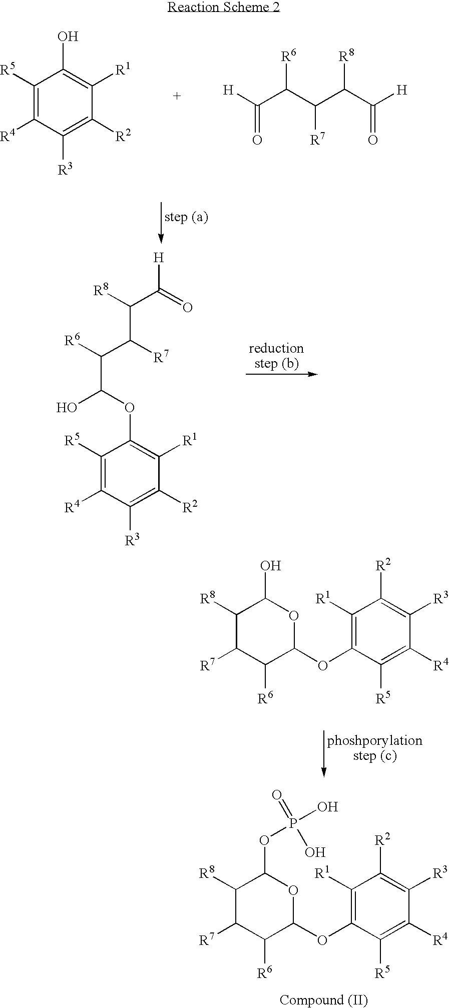

In one preferred embodiment, the product of step (c) is further reacted with a complexing agent selected from the group comprising amphoteric surfactants, cationic surfactants, amino acids having nitrogen functional groups and proteins rich in these amino acids.

According to a second aspect of the invention, there is provided a method for preparing a phosphate derivative of a phenolic hydroxy compound comprising the following steps:
(a) reacting a phenolic hydroxy compound with an alkyl (X: dialdehyde or a sugar-like polyhydroxy dialdehyde to form a hemiacetal;
(b) reducing the terminal aldehyde group on the product from step (a) to a hydroxyl group; and
(c) phosphorylating the hydroxyl group formed in step (b).

In one preferred embodiment, the method further comprises step (d) reacting the product of step (c) with a complexing agent selected from the group comprising amphoteric surfactants, cationic surfactants, amino acids having nitrogen functional groups and proteins rich in these amino acids.

According to a third aspect of the invention, there is provided a method for improving the bioavailability of a phenolic hydroxy compound comprising the following steps:
(a) reacting the phenolic hydroxy compound with an alkyl o:c dialdehyde or a sugar-like polyhydroxy dialdehyde to form a hemiacetal;
(b) reducing the terminal aldehyde group on the product from step (a) to a hydroxyl group; and
(c) phosphorylating the hydroxyl group formed in step (b) to produce a phosphate derivative of the phenolic hydroxy compound.

Where used herein the term “phosphate derivatives” refers to compounds covalently bound by means of an oxygen to the phosphorus atom of a phosphate group. The phosphate derivative may exist in the form of a free phosphate acid, a salt thereof, a di-phosphate ester thereby including two phenolic hydroxy compound molecules, a mixed ester including one phenolic hydroxy compound and another phenolic hydroxy compound, and a phosphatidyl compound wherein the free phosphate oxygen forms a bond with an alkyl or substituted alkyl group. Suitable complexing agents for use in the invention may be selected surfactants chosen from classes including from alkyl amino/amido betaines, sultaines, phosphobetaines, phosphitaines, imidazolimum and straight chain mono and dicarboxy ampholytes, quaternary ammonium salts, and cationic alkoxylated mono and di-fatty amines; and amino acids having nitrogen functional groups and proteins rich in these amino acids. Preferred complexing agents are N-lauryl imino di-propionate and arginine.

Suitable amino acids having nitrogen functional groups for use in the invention include glycine, arginine, lysine and histidine. Proteins rich in these amino acids may also be used as complexing agents, for example, casein. These complexing agents are used when the composition needs to be delivered by other routes of administration including but not limited to inhalation, oral ingestion, dermal application, eye drops or suppositories.

The amphoteric surfactants may be ampholytic surfactants, that is, they exhibit a pronounced isoelectric point within a specific pH range; or zwitterionic surfactants, that is, they are cationic over the entire pH range and do not usually exhibit a pronounced isoelectric point. Examples of these amphoteric surfactants are tertiary substituted amines, such as those according to the following formula:
NR⁹R¹⁰R¹¹

wherein R⁹ is chosen from the group comprising straight or branched chain mixed alkyl radicals from C6 to C22 and carbonyl derivatives thereof.

R¹⁰ and R¹¹ are independently chosen from the group comprising H, CH₂COOX, CH₂CHOHCH₂SO₃X, CH₂CHOHCH₂OPO₃X, CH₂CH₂COOX, CH₂COOX, CH₂CH₂CHOHCH₂SO₃X or CH₂CH₂CHOHCH₂OPO₃X and X is H, Na, K or alkanolamine provided that R¹⁰ and R¹¹ are not both H.

In addition, when R⁹ is RCO then R¹⁰ may be CH₃ and R¹¹ may be (CH₂CH₂)N(C₂HOH)—H₂COPO₃ or R¹⁰ and R¹¹ together may be N(CH₂)₂N(C₂H₄OH)CH₂COO—.

Commercial examples are DERIPHAT sold by Henkel/Cognis, DEHYTON sold by Henkel/Cognis, TEGOBETAINE sold by Goldschmidt and MIRANOL sold by Rhone Poulenc.

Cationic surfactants, such as quaternary ammonium compounds, will also form complexes with phosphorylated derivatives of drug hydroxy compounds such as tocopheryl phosphates. Examples of cationic surfactants include the following:
(a) RN⁺(CH₃)₃Cl⁻
(b) [R₂N⁺CH₃)₂SO₄²⁻
(c) RCON(CH₃)CH₂CH₂CH₂N⁺(CH₃)₂C₂H₄OH]₂ SO₄²⁼³¹
(d) Ethomeens: RN[(CH₂CH₂O), CH₂OH][(CH₂CH₂O)_{y} CH₂OH] wherein x and y are independently integers from 1 to 50.

wherein R is C8 to C22 straight or branched chain alkyl groups or mixed alkyl groups.

Silicone surfactants including hydrophilic and hydrophobic functionality may also be used, for example, dimethicone PG betaine, amodimethicone or trimethylsilylamodimethicone. For example, ABILE 9950 from Goldschmidt Chemical Co. The hydrophobe can be a C6 to C22 straight -or branched alkyl or mixed alkyl including fluoroalkyl, fluorosilicone and or mixtures thereof. The hydrophilic portion can be an alkali metal, alkaline earth or alkanolamine salts of carboxy alkyl groups or sulfoxy alkyl groups, that is sultaines, phosphitaines or phosphobetaines or mixtures thereof.

Typically, the complex of the phosphate derivative of the phenolic hydroxy compound is made by (1) direct neutralization of the free phosphoric acid ester of the phenolic hydroxy compound with the complexing agents or (2) in-situ blending of mixed sodium salts of the phosphate derivatives of the phenolic hydroxy compound with the complexing agents.

Propofol is an example of a phenolic hydroxy compound to which the invention may have application. Forms of propofol which may be used in this invention include:

2,6-diisopropylphenol (CAS 2078-54-8)

Propofol phosphate or Phenol, 2,6-bis(1-methylethyl)-, dihydrogen phosphate (9CI) (CAS 18351-38-7)

Phenol, 2,6-bis(1-methylethyl)-, dihydrogen phosphate, disodium salt (9CI) (CAS 250345-80-3)

Adrenaline and analgesics are examples of other phenolic hydroxy compounds which may be used in the invention.

### EXAMPLES

The invention will now be further explained and illustrated by reference to the following non-limiting examples.

### Example 1

### Preparation of Phosphate Derivative of Propofol

17.8 g (0.1M) of 2,6-diisopropylphenol (propofol) was placed in a 100 ml flask with a good agitator. 4.2 g of sodium hydrogen carbonate and 3.4 g of sodium carbonate were dissolved in 23.2 g of 50% aqueous gluteraldehyde. This solution was added to the 2,6-disopropylphenol with vigorous stirring over a one hour period. Then stirring continued for one hour. The water was evaporated to give the dry hemiacetal derivative of 2,6-diisopropylphenol (A). A was dissolved in 50 ml of toluene, then 7.8 g of P₄O₁₀ was added and the mixture stirred for one hour with the temperature maintained in the range 40 to 60° C. 50 ml of water was carefully added and the mixture stirred for thirty minutes to hydrolyse any pyrophosphates. The toluene phase was separated using a separating funnel and dried to produce 2-(2,6-diisopropylphenoxy)-tetrahydropyran-6-yl , dihydrogen phosphate (1).

### Example 2

### Preparation of Phosphate Derivative of Propofol

17.8 g (0.1M) of 2,6-diisopropylphenol (propofol) was placed in a 100 ml flask with a good agitator. 4.2 g of sodium hydrogen carbonate and 3.4 g of sodium carbonate were dissolved in 32.6 g of 50% aqueous trihydroxy pentandial. This solution was added to the 2,6-di-isopropylphenol with vigorous stirring over a one hour period. Then stirring continued for one hour. The water was evaporated to give the dry hemiacetal derivative of 2,6-diisopropylphenol (B). B was dissolved in 50 ml of toluene, then 7.8 g of P₄O₁₀ was added and the mixture stirred for one hour, maintaining the temperature in the range 40 to 60° C. 50 ml of water was carefully added and the mixture stirred for thirty minutes to hydrolyse any pyrophosphates. The toluene phase was separated using a separating funnel and dried to produce 2-(2,6-diisopropylphenoxy)-3,4,5-trihydroxy tetrahydropyran-6-yl, dihydrogen phosphate (II).

### Example 3

### Preparation of Phosphate Derivative of Propofol

17.8 g (0.1M) of 2,6-diisopropylphenol (propofol) was placed in a 100 ml flask with a good agitator. 4.2 g of sodium hydrogen carbonate and 3.4 g of sodium carbonate were dissolved in 12.8 g of 50% aqueous glyoxyal. This solution was added to the 2,6-diisopropylphenol with vigorous stirring over a one hour period. Then stirring continued for one hour. 3.8 g of sodium borohydride was added and the mixture stirred for one hour. The water was evaporated to give the dry hemiacetal derivative of 2,6-diisopropylphenol (C). C was dissolved in 50 ml of toluene, then 7.8 g of P₄O₁₀ was added and the mixture stirred for one hour, maintaining the temperature in the range 40 to 60° C. 25 ml of water was carefully added and the mixture stirred for thirty minutes to hydrolyse any pyrophosphates. The toluene phase was separated using a separating funnel and dried to produce 2-(2,6-diisopropylphenoxy)-2-hydroxy ethylphosphate (III).

### Example 4

### Preparation of Complex of Phosphate Derivative of Propofol

373 g (1 M) of disodium lauryl-imino-dipropionate was dissolved in 2000 ml of deionized water and warmed to 50-60° C. to form a clear solution of pH 11-12. 358 g (1 M) of product I from Example 1 was added with good agitation to form the disodium lauryl-imino-dipropionate-2-(2,6-diisopropylphenoxy) tetrahydropyran-6-yl dihydrogen phosphate complex (IV) at a pH of 8-9 as an aqueous solution. The pH may be adjusted by adding appropriate amounts of either component.

### Example 5

### Preparation of Complex of Phosphate Derivative of Propofol

174 grams of arginine was dissolved in 1000 ml of deionized water. 406 g of product II from Example 2 was added to this solution with good agitation to yield the arginine 2-(2,6-diisopropylphenoxy)-3,4,5-trihydroxy tetrahydropyran-6-yl dihydrogen phosphate complex (V) as an aqueous solution with final pH of 6.5-7.5.

### Example 6

### Preparation of Complex of Phosphate Derivative of Propofol

17 g (0.1M) arginine was dissolved into 100 ml of deionized water. 31.8 g (0.1M) of product III from Example 3 was added to this solution with good agitation to form an arginine 2-(2,6-diisopropylphenoxy)-2-hydroxy ethylphosphate complex (VI) as an aqueous complex with final pH of 6.5-7.5.

### Example 7

### Preparation of Complex of Phosphate Derivative of Propofol

373 g (1 M) of disodium lauryl-imino-dipropionate was dissolved in 200 ml of deionized water and warmed to 50-60° C. to form a clear solution of pH 11-12. 358 g (1 M) of product I from Example 1 was added with good agitation to form the disodium lauryl-imino-dipropionate 2-(2,6-diisopropylphenoxy) tetrahydropyran-6-yl dihydrogen phosphate complex (VII) at a pH of 8-9 as an aqueous solution. The pH may be adjusted by adding appropriate amounts of either component. The solution was then freeze dried for 24 hours to yield the complex as a dry powder.

### Example 8

### Preparation of Complex of Phosphate Derivative of Propofol

174 grams of arginine was dissolved in 200 ml of deionized water. 406 g of product II from Example 2 was added to this solution with good agitation to yield the 2-(2,6-diisopropylphenxoy)-3,4,5-trihydroxytetrahydropyran-6-yl dihydrogen phosphate arginine complex (VIII) with final pH of 6.5-7.5. The solution was then freeze dried for 24 hours to yield the complex as a dry powder.

### Example 9

### Preparation of Complex of Phosphate Derivative of Propofol

17 g (0.1M) arginine was dissolved into 20 ml of deionized water. 31.8 g (0.1M) of product III from Example 3 was added with good agitation to form an arginine 2-(2,6-diisopropylphenoxy)-2-hydroxy ethylphosphate complex (IX) as an aqueous complex with final pH of 6.5-7.5. The solution was then freeze dried for 24 hours to yield the complex as a dry powder.

### Example 10

### Preparation of Complex of Phosphate Derivative of Propofol

174 grams of arginine was dissolved in 200 ml of deionized water. 358 g of product I from Example 1 was added to this solution with good agitation to yield the 2-(2,6-diisopropylphenoxy)-tetrahydropyran-6-yl dihydrogen phosphate arginine complex (X) with final pH of 6.5-7.5. 0.3M 2,6-di-isopropylphenol was added and fully emulsified with a high sheer agitator. The solution was then freeze dried for 24 hours to yield a product being a mixture of the complex and free 2,6-diisopropylphenol that when used intravenously acted as an anaesthetic. The free 2,6-diisopropylphenol was available for immediate anaesthetic action in an emulsified state and the complex for slower delivery of the 2,6-diisopropylphenol after hydrolysis.

The word ‘comprising’ and forms of the word ‘comprising’ as used in this description and in the claims does not limit the invention claimed to exclude any variants or additions.

Modifications and improvements to the invention will be readily apparent to those skilled in the art. Such modifications and improvements are intended to be within the scope of this invention.

1. A phosphate derivative of a phenolic hydroxy compound comprising the reaction product of the following steps:
(a) reacting the phenolic hydroxy compound with an alkyl α:ω dialdehyde or a sugar-like polyhydroxy dialdehyde to form a hemiacetal;
(b) reducing the terminal aldehyde group on the product from step (a) to a hydroxyl group; and
(c) phosphorylating the hydroxyl group formed in step (b) to produce a phosphate derivative of the phenolic hydroxy compound.

2. The phosphate derivative of a phenolic hydroxy compound according to claim 1 having the structure of Compound (I) wherein R¹, R², R³, R⁴ and R⁵ may each independently be chosen from H or an alkyl group and n and m are independently in the range of 0 to 8.

3. The phosphate derivative of a phenolic hydroxy compound according to claim 1 having the structure of Compound (II) wherein R¹, R², R³, R⁴ and R⁵ may each independently be chosen from H or an alkyl group and R⁶, R⁷ and R⁸ can each independently be H or OH.

4. The phosphate derivative of a phenolic hydroxy compound according to claim 1 wherein the product of step (c) has been reacted with a complexing agent selected from the group comprising amphoteric surfactants, cationic surfactants, amino acids having nitrogen functional groups and proteins rich in these amino acids.

5. The phosphate derivative of a phenolic hydroxy compound according to claim 1 wherein the phenolic hydroxy compound is propofol or a derivative of propofol.

6. The phosphate derivative of a phenolic hydroxy compound according to claim 5 wherein the phosphate derivative of propofol has been reacted with a complexing agent selected from the group comprising amphoteric surfactants, cationic surfactants, amino acids having nitrogen functional groups and proteins rich in these amino acids.

7. The phosphate derivative of a phenolic hydroxy compound according to claim 6 wherein the complexing agent is arginine.

8. The phosphate derivative of a phenolic hydroxy compound according to claim 6 wherein the complexing agent is disodium lauryl-imino-dipropionate.

9. The phosphate derivative of a phenolic hydroxy compound according to claim 1 wherein the alkyl x:(o dialdehyde or a sugar-like polyhydroxy dialdehyde is selected from the group consisting of gluteraldehyde, trihydroxy pentandial, glyoxyal and mixtures thereof.

10. The phosphate derivative of a phenolic hydroxy compound of claim 1 wherein the phenolic hydroxy compound is selected from adrenaline, analgesics and mixtures thereof.

11. A method for preparing a phosphate derivative of a phenolic hydroxy compound comprising the steps of:
(a) reacting the phenolic hydroxy compound with an alkyl α:ω dialdehyde or a sugar-like polyhydroxy dialdehyde to form a hemiacetal;
(b) reducing the terminal aldehyde group on the product from step (a) to a hydroxyl group; and
(c) phosphorylating the hydroxyl group formed in step (b) to produce a phosphate derivative of the phenolic hydroxy compound.

12. The method according to claim 11 further comprising step (d) reacting the product of step (c) with a complexing agent selected from the group comprising amphoteric surfactants, cationic surfactants, amino acids having nitrogen functional groups and proteins rich in these amino acids.

13. The method according to claim 11 wherein the phenolic hydroxy compound is propofol or a derivative of propofol.

14. The method according to claim 13 comprising the further step of reacting the phosphate derivative of propofol with a complexing agent selected from the group comprising amphoteric surfactants, cationic surfactants, amino acids having nitrogen functional groups and proteins rich in these amino acids.

15. The method according to claim 14 wherein the complexing agent is arginine.

16. The method according to claim 14 wherein the complexing agent is disodium lauryl-imino-dipropionate.

17. The method according to claim 11 wherein the alkyl α:ω dialdehyde or a sugar-like polyhydroxy dialdehyde is selected from the group consisting of gluteraldehyde, trihydroxy pentandial, glyoxyal and mixtures thereof.

18. A phosphate derivative of propofol or a derivative of propofol comprising the reaction product of the following steps:
(a) reacting propofol or a derivative of propofol with an alkyl o:( dialdehyde or a sugar-like polyhydroxy dialdehyde to form a hemiacetal;
(b) reducing the terminal aldehyde group on the product from step (a) to a hydroxyl group; and
(c) phosphorylating the hydroxyl group formed in step (b) to produce a phosphate derivative of propofol or a derivative of propofol.

19. The phosphate derivative of propofol or a derivative of propofol according to claim 18 wherein the phosphate derivative from step (c) has been reacted with a complexing agent selected from the group comprising amphoteric surfactants, cationic surfactants, amino acids having nitrogen functional groups and proteins rich in these amino acids.

20. The phosphate derivative of propofol or a derivative of propofol according to claim 19 wherein the complexing agent is arginine.

21. The phosphate derivative of propofol or a derivative of propofol according to claim 19 wherein the complexing agent is disodium lauryl-imino-dipropionate.

22. The phosphate derivative of propofol or a derivative of propofol according to claim 18 wherein the alkyl a:o) dialdehyde or a sugar-like polyhydroxy dialdehyde is selected from the group consisting of gluteraldehyde, trihydroxy pentandial, glyoxyal and mixtures thereof.

23. A phosphate derivative of a phenolic hydroxy compound according to claim 1 when used as a prodrug.

24. A phosphate derivative of a phenolic hydroxy compound according to claim 1 when used as an anaesthetic.

25. A method for improving the bioavailability of a phenolic hydroxy compound comprising the following steps:
(a) reacting the phenolic hydroxy compound with an alkyl α:ω dialdehyde or a sugar-like polyhydroxy dialdehyde to form a hemiacetal;
(b) reducing the terminal aldehyde group on the product from step (a) to a hydroxyl group; and
(c) phosphorylating the hydroxyl group formed in step (b) to produce a phosphate derivative of the phenolic hydroxy compound.
